(19) United States
(12) Patent Application Publication
(10) Pub. No.: US 2015/0141287 A1
(43) Pub. Date: May 21, 2015

(54) GENETIC MARKERS ASSOCIATED WITH INTELLECTUAL DISABILITY

(71) Applicant: CENTRE FOR ADDICTION AND MENTAL HEALTH, Toronto (CA)

(72) Inventor: John B. Vincent, Toronto (CA)

(21) Appl. No.: 14/602,632

(22) Filed: Jan. 22, 2015

Related U.S. Application Data
(63) Continuation of application No. 13/805,884, filed on Jan. 17, 2013.
(60) Provisional application No. 61/357,657, filed on Jun. 23, 2010.

(51) Int. Cl. C12Q 1/68; C07K 16/40; C12N 9/10

(52) U.S. Cl. CPC C12Q 1/6883; C12N 9/1007; C07K 16/40; C12Q 2600/156; C12Y 201/01203
USPC 506/9; 536/24.31; 435/6.11; 536/23.2; 435/193; 530/387.9; 506/16

(57) ABSTRACT

Genetic markers associated with intellectual disability as well as compositions, methods and kits for screening for genetic markers of intellectual disability, diagnosing intellectual disability and identifying individuals with a predisposition for offspring suffering from intellectual disability are provided.

This patent application in a continuation of U.S. application Ser. No. 13/805,884, filed Jan. 17, 2013, which is the National Stage of International Application No. PCT/CA2011/050388 filed Jun. 23, 2011, which claims the benefit of priority from U.S. Provisional Application Ser. No. 61/357,657, filed Jun. 23, 2010, teachings of each of which are hereby incorporated by reference in their entirety.

### FIELD OF THE INVENTION

The present invention relates to genetic diagnosis of intellectual disability. In particular, the present invention relates to a genetic marker associated with intellectually disabled individuals and methods and kits for use of these markers.

### BACKGROUND OF THE INVENTION

Intellectual disability, also called mental retardation, is a devastating neurodevelopmental disorder with serious impact on the affected individuals and their families, as well as on health and social services. It is believed to occur with a prevalence of approximately 1-3% within the population, and is frequently the result of genetic aberrations. Intellectual disability may present as the sole clinical feature (non-syndromic), or may be present with additional clinical or dysmorphological features (syndromic). Intellectual disability is significantly more frequent in males than in females and it had been assumed that approximately 25% of severe cases were X-linked. A recent review, however, suggests that X-linked mutations contribute to no more than 10% of cases (Ropers, H. H. and Hamel, B. C. Nat Rev Genet 2005 6:46-57). Little is known about autosomal non-syndromic forms of intellectual disability.

Autosomal recessive forms of non-syndromic intellectual disability are believed to be more common. To date, 6 autosomal recessive genes for non-syndromic forms of intellectual disability have been reported.

A locus was identified as harboring a gene for non-syndromic autosomal recessive mental retardation in 78 consanguineous Iranian families (Najmabadi et al. Hum Genet 2007; 121:43-48). No causative gene was reported.

A number of additional loci have also been mapped.

### SUMMARY OF THE INVENTION

The present invention relates to identification of a gene in human chromosomal locus 5p15.32-p15.31 associated with intellectual disability.

Thus, an aspect of the present invention relates to identification of mutations in the NSUN2 gene located at chromosomal locus 5p15.32-p15.31 associated with intellectual disability.

In one embodiment, the present invention relates to a point mutation in exon 19 of the NSUN2 gene resulting in a missense mutation in the encoded protein associated with intellectual disability.

In one embodiment, the present invention relates to a homozygous base substitution exon 19 of the NSUN2 gene resulting in a missense mutation in the encoded protein associated with intellectual disability.

In one embodiment, the present invention relates to a homozygous G>A substitution (see SEQ ID NO:1 and 2 and FIGS. 9 and 10, respectively) corresponding to nucleotide position 2035 from the translation start site in the mRNA of GENBANK sequence accession no. AK291144 (depicted in SEQ ID NO:1 and FIG. 9) associated with intellectual disability.

In one embodiment, the present invention relates to a missense mutation Gly679Arg (see SEQ ID NO:3 and 4 and FIGS. 11 and 12, respectively) in the amino acid sequence encoded by the NSUN2 gene associated with intellectual disability.

Another aspect of the present invention relates to isolated polynucleotides and polypeptides encoded thereby comprising a genetic marker for intellectual disability.

In one embodiment, the marker is the NSUN2 gene located at chromosomal locus 5p15.32-p15.31.

In one embodiment, the marker is exon 19 of the NSUN2 gene located at chromosomal locus 5p15.32-p15.31.

In one embodiment, the marker is a homozygous G>A substitution (see SEQ ID NO: 1 and 2 and FIGS. 9 and 10, respectively) corresponding to nucleotide position 2035 from the translation start site in the mRNA of GENBANK sequence accession no. AK291144 (depicted in SEQ ID NO:1 and FIG. 9).

In one embodiment, the marker is a missense mutation Gly679Arg (see SEQ ID NO: 3 and 4 and FIGS. 11 and 12, respectively) in the amino acid sequence of GENBANK sequence accession no. AK291144 (depicted in SEQ ID NO:3 and FIG. 11).

Another aspect of the present invention relates to a method of screening an individual for a genetic marker associated with intellectual disability.

In one embodiment, the method comprises analyzing exon 19 of the NSUN2 gene located at chromosomal locus 5p15.32-p15.31 in an individual for a mutation.

In one embodiment, the method comprises analyzing an individual for a homozygous G>A substitution (see SEQ ID NO:1 and 2 and FIGS. 9 and 10, respectively) corresponding to nucleotide position 2035 from the translation start site in the mRNA of GENBANK sequence accession no. AK291144 (depicted in SEQ ID NO:1 and FIG. 9).

In one embodiment, the method comprises analyzing an individual for a missense mutation Gly679Arg (see SEQ ID NO:3 and 4 and FIGS. 11 and 12, respectively) in the amino acid sequence of GENBANK sequence accession no. AK291144 (depicted in SEQ ID NO:3 and FIG. 11).

Another aspect of the present invention relates to compositions and kits useful in these screening methods.

Another aspect of the present invention relates to a method of genetically diagnosing intellectual disability in an individual.

Another aspect of the present invention relates to compositions and kits useful in methods of genetically diagnosing intellectual disability in an individual.

Another aspect of the present invention relates to a method for identifying individuals predisposed genetically to offspring suffering from intellectual disability.

Another aspect of the present invention relates to compositions and kits useful in methods for identifying individuals predisposed genetically to offspring suffering from intellectual disability.

Other and further objects, features, and advantages will be apparent and readily understood by reading the following specification and by reference to the accompanying drawings forming a part thereof, or any examples of the presently preferred embodiments of the invention given for the purpose of the disclosure.

### BRIEF DESCRIPTION OF THE DRAWINGS

FIG. 1 is a diagram of the pedigree structure of patients studied. Filled circles indicate affected girls.

FIG. 2 is a graph showing results from genome wide Homozygosity Mapper analysis for microarray single nucleotide polymorphism data, genome-wide. Significant regions of homozygosity-by-descent (HBD) were seen only on 5p and 14q. The 14q locus was excluded because one of the unaffected siblings was also homozygous at this locus, whereas at the 5p locus, unaffected the sibling was genotyped as heterozygous.

FIG. 3 is a graph showing results from Homozygosity Mapper analysis of microarray single nucleotide polymorphism data of chromosome 5.

FIG. 4 is an ideogrammatic representation of the critical autozygous or HBD locus on 5p15.31, as determined herein and in relation to the MRT5 locus identified by Najmabadi et al. (Hum Genet 2007; 121:43-48 (2007).

FIGS. 5A through 5D are photomicrographs of the human breast cancer cell line HCC1954 24 hours after transfection with wild type (WT) NSUN2 (FIG. 5A) or the mutant construct NSUN2-G679R (FIGS. 5B, 5C and 5D). Cells were stained with antibodies to the Myc epitope in order to detect transfected proteins. DAPI staining was used to show nuclear localization.

FIGS. 6A and 6B are photomicrographs of the human breast cancer cell line HCC1954 24 hours after transfection with wild type (WT) NSUN2 (FIG. 6A) or the mutant construct NSUN2-G679R (FIG. 6B) stained with antibodies to the nucleolar marker protein, nucleophosmin (NPM1) to confirm co-localization in the nucleoli. Quantification of co-localization is depicted in the bar graph of FIG. 6C.

FIGS. 7A and 7B shows photomicrographs of the human breast cancer cell line HCC1954 24 hours after transfection with wild type (WT) NSUN2 (FIG. 7A) or the mutant construct NSUN2-G679R (FIG. 7B) co-stained with the proliferating cell nuclear antigen (PCNA).

FIGS. 8A through 8C are photomicrographs of COS7 cells 24 hours after transfection with wild type (WT) NSUN2 (FIG. 8A) or the mutant construct NSUN2-G679R (FIGS. 8B and 8C) stained with antibodies to the nucleolar marker protein, nucleophosmin (NPM1) to confirm co-localization in the nucleoli. DAPI staining was used to show nuclear localization.

FIG. 9 shows the nucleic acid sequence of wild-type Homo sapiens cDNA FLJ76184 of GenBank Accession No. AK291144 (SEQ ID NO:1). The complete coding sequence, highly similar to Homo sapiens NOL1/NOP2/Sun domain family, member 2 (NSUN2), mRNA, is depicted. The start ATG codon is indicated by italicized capitol letters. The nucleotide identified to be mutated from G>A in intellectual disability is indicated by bolding and underlining.

FIG. 10 shows an isolated polynucleotide of the present invention with a missense mutation associated with intellectual disability (SEQ ID NO:2). The start ATG codon is indicated by italicized capitol letters. The nucleotide identified to be mutated from G>A in intellectual disability is indicated by bolding and underlining.

FIG. 11 shows the amino acid sequence (SEQ ID NO:3) of the protein encoded by the nucleic acid sequence (SEQ ID NO:1) of FIG. 9.

FIG. 12 shows the amino acid sequence (SEQ ID NO:4) of an isolated polypeptide of the present invention encoded by the polynucleotide (SEQ ID NO:2) of FIG. 10.

### DETAILED DESCRIPTION OF THE INVENTION

A gene has now been identified on the human chromosomal locus 5p15.32-p15.31, associated with intellectual disability. The present invention provides genetic markers for intellectual disability related to this gene comprising isolated polynucleotides and polypeptides encoded thereby, as well as methods, compositions and kits for genetically diagnosing intellectual disability in an individual and identifying individuals predisposed genetically to offspring suffering from intellectual disability.

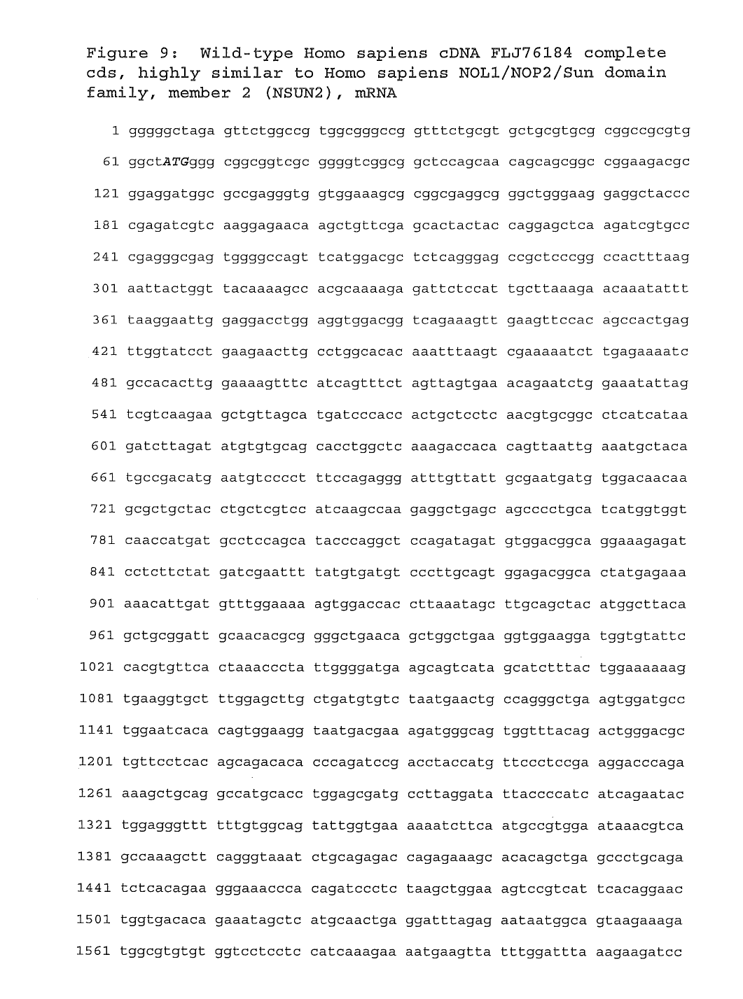

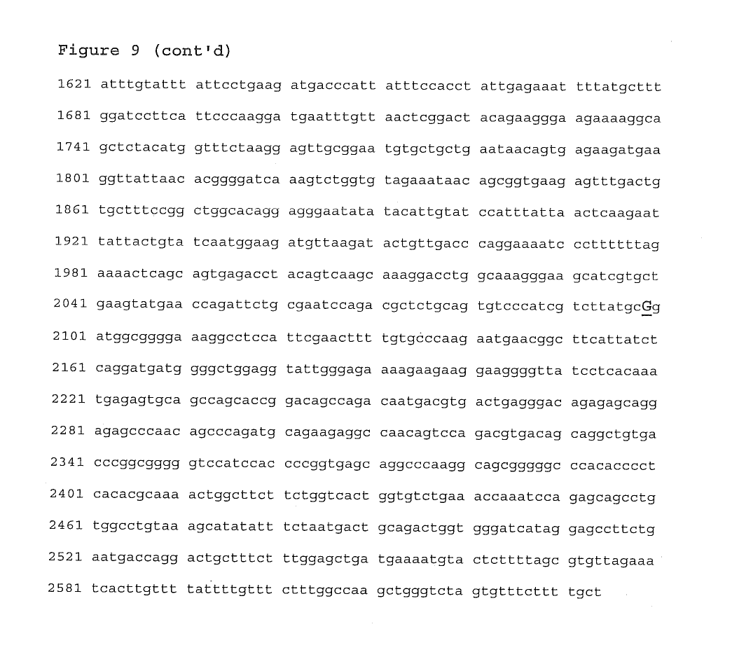

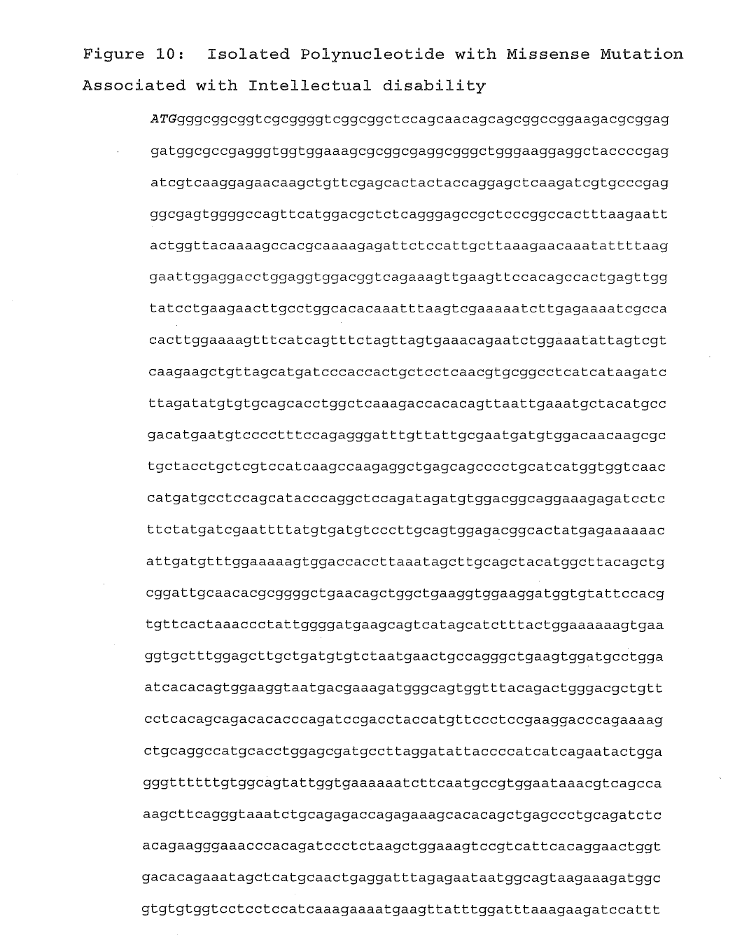

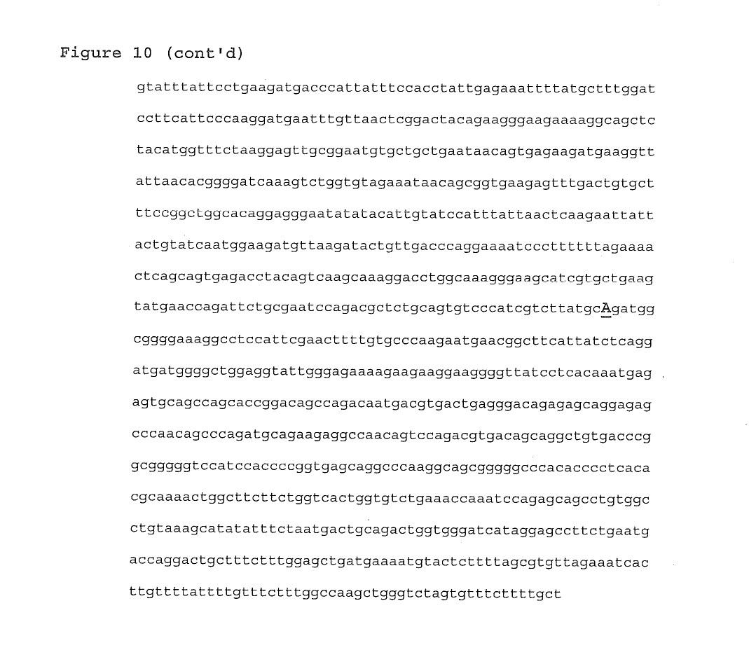

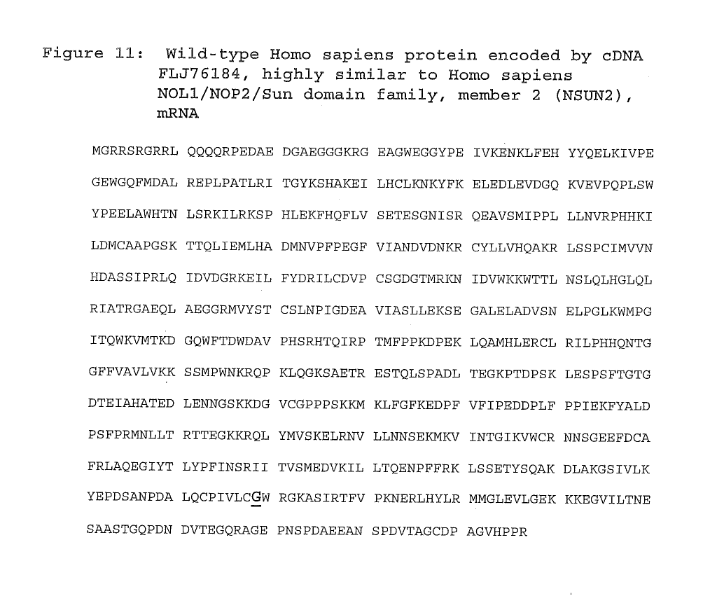

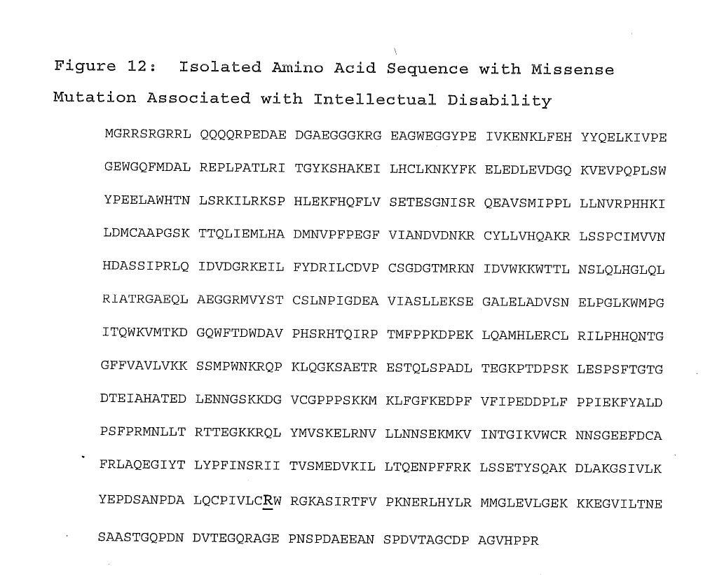

In particular, a polymorphism associated with intellectual disability has now been identified in exon 19 of the NSUN2 gene. The polymorphism corresponds to nucleotide position 2035 from the translation start site in the mRNA of GENBANK sequence accession no. AK291144 (also depicted herein as nucleotide 2099 of SEQ ID NO:1 and FIG. 9 and SEQ ID NO:2 and FIG. 10). The polymorphism comprises a homozygous G>A base substitution resulting in a missense mutation Gly679Arg in the encoded amino acid sequence (see SEQ ID NO:3 and FIG. 11 and SEQ ID NO:4 and FIG. 12).

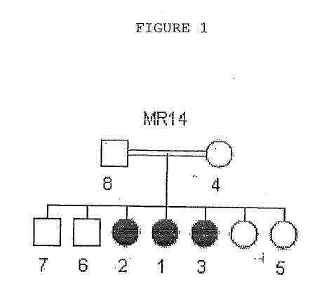

The single nucleotide polymorphism of the present invention was identified in a consanguineous family from Pakistan multiplex with non-syndromic intellectual disability. Pedigree analysis as depicted in FIG. 1 indicated that the condition segregates in an autosomal recessive pattern. Clinical studies of 3 individuals of the family affected with non-syndromic intellectual disability (described in more detail in Example 2) indicated the presence of a distal myopathy as well as some possible dysmorphic features such as mild neck webbing.

Microarray analysis using Affymetrix 250K NspI arrays was performed on affected family members. A 2.7 Mb region was identified on 5p15.32-p15.31 with a continuous run of 798 single nucleotide polymorphisms homozygous common among all affecteds in the family.

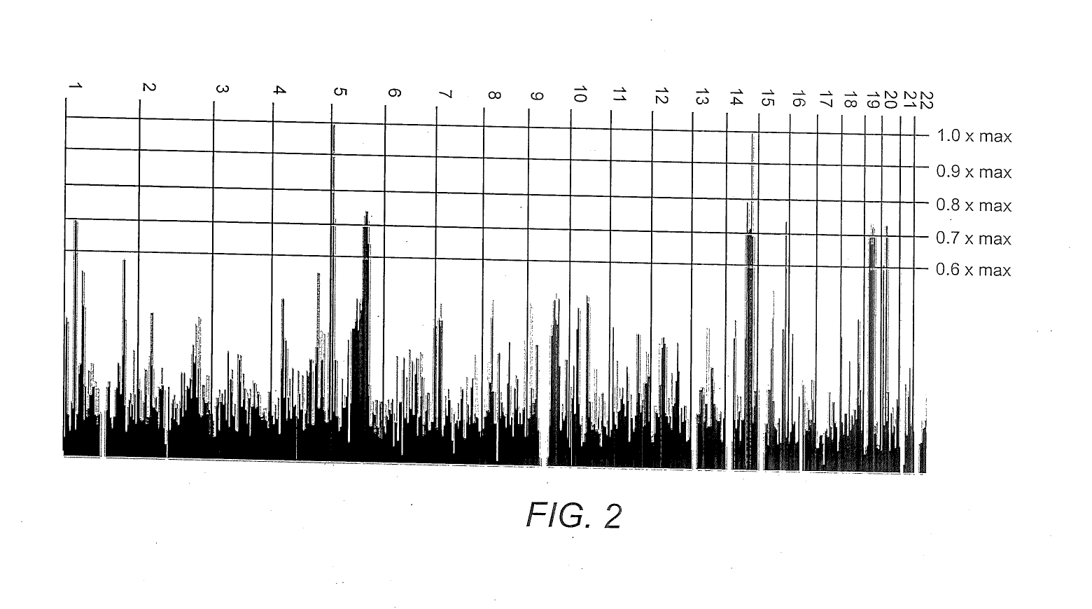

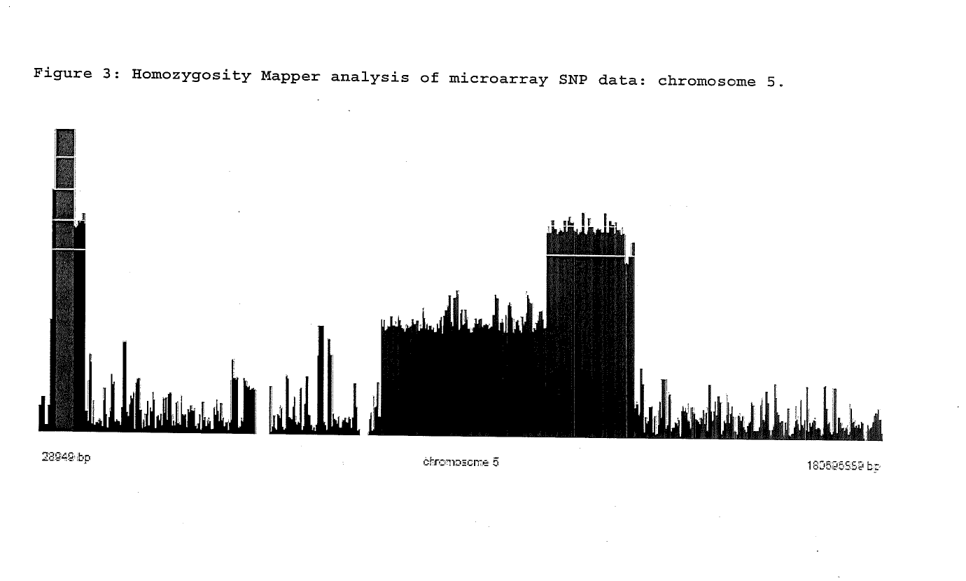

Homozygosity mapper analysis of the microarray single nucleotide polymorphism data is depicted in FIGS. 2 and 3. FIG. 2 shows homozygosity mapper analysis of the entire genome. Significant regions of homozygosity-by-descent were seen only on 5p and 14q. However, the 14q locus was excluded because one of the unaffected siblings was also homozygous at this locus. At the 5p locus, the unaffected sibling was genotyped as heterozygous. Accordingly, FIG. 3 shows data from homozygosity mapper analysis of microarray single nucleotide polymorphism data for the 5p locus only.

Additional genotype data from microsatellite markers on 5p verified findings from the microarray analysis. Two-point linkage analysis produced a lod score of 2.77. These data are depicted in Table 1.

TABLE 1
Two-point linkage analysis for markers across
chromosome 5p

| | | Phy- | LOD Score at Recombination | | | |
|---|---|---|---|---|---|---|
| | | Pos | Fraction, θ | | | |
| Markers | Pos-cM | Mb | 0.00 | 0.05 | 0.2 | 0.4 |
| D5S1981 | 1.7200 | 1.155 | 0.4327 | 0.3972 | 0.2597 | 0.0585 |
| D5S406 | 11.8500 | 4.994 | 2.7729 | 2.4887 | 1.6231 | 0.5141 |
| D5S2505 | 14.3000 | 5.817 | 1.6695 | 1.4721 | 0.8628 | 0.1437 |
| D5S580 | 17.8700 | 8.141 | 0.4327 | 0.3972 | 0.2597 | 0.0585 |
| D5S630 | 19.6710 | 9.561 | 0.4327 | 0.3972 | 0.2597 | 0.0585 |

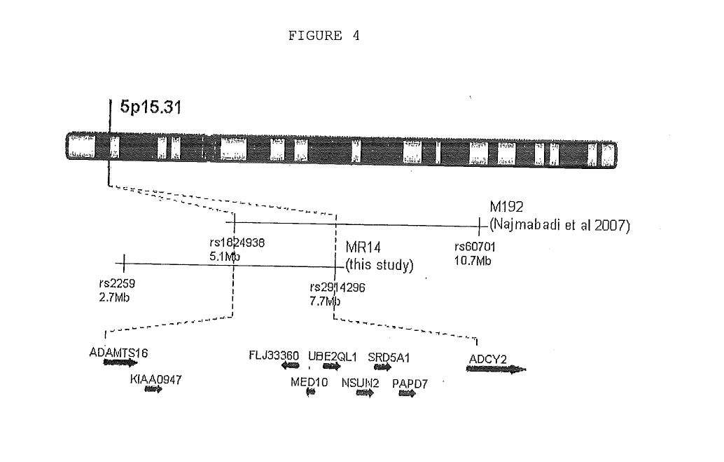

Physical position was selected according to UCSC February 2009 (GRCh37/hg19) assembly. For reference, the common homozygosity-by-descent locus for the Pakistani family and for the Iranian family M192 studied by Najmabadi et al. (Hum Genet 2007; 121:43-48) extends from single nucleotide polymorphisms rs1824938 (5.092 Mb) to rs2914296 (7.658 Mb). NSUN2 is located between 6.599 and 6.633 Mb (see FIG. 4).

All known coding genes within this region were screened for mutations by sequencing. A homozygous G>A base substitution at nucleotide position 2035 (from the translation start site in mRNA of GENBANK sequence accession no. AK291144) was detected within exon 19 of the gene NSUN2, one of 8 known genes within this locus. To the best of the inventors' knowledge, this substitution is not a known single nucleotide polymorphism in any single nucleotide polymorphism database. Further, this mutation was not present in over 400 chromosomes from Pakistani control individuals.

A 250 bp insertion just following exon 9 was also identified. However, after genotyping Pakistani controls, it was apparent that this is a relatively common polymorphism.

NSUN2 encodes a methyltransferase that catalyzes the intron-dependent formation of 5-methylcytosine at C34 of tRNA-leu(CAA) (Brzezicha et al. Nucleic Acids Res 2006 34:6034-6043). It also functions in spindle assembly during mitosis as well as chromosome segregation (Hussain et al. J Cell Biol 2009 186:27-40).

The homozygous G>A base substitution at nucleotide position 2035 results in a missense mutation, Gly679Arg in the encoded amino acid sequence. This amino acid residue appears to be conserved across the animal and plant kingdoms. The Gly679Arg substitution does not appear to be known single nucleotide polymorphism and to the best of the inventor's knowledge has not been identified to date in any databases for single nucleotide polymorphisms. The Gly679Arg substitution was also not present in over 400 chromosomes from Pakistani control individuals.

The NSUN2 protein carrying the Gly679Arg missense mutation was found to function incorrectly when transfected into cells thus establishing a distinct cellular phenotype for this missense mutation from wild type NSUN2 (WT NSUN2). While the wild type construct correctly localized to the nucleolus, the correct cellular location for NSUN2 protein, the Gly679Arg mutant NSUN2 was excluded from the nucleolus.

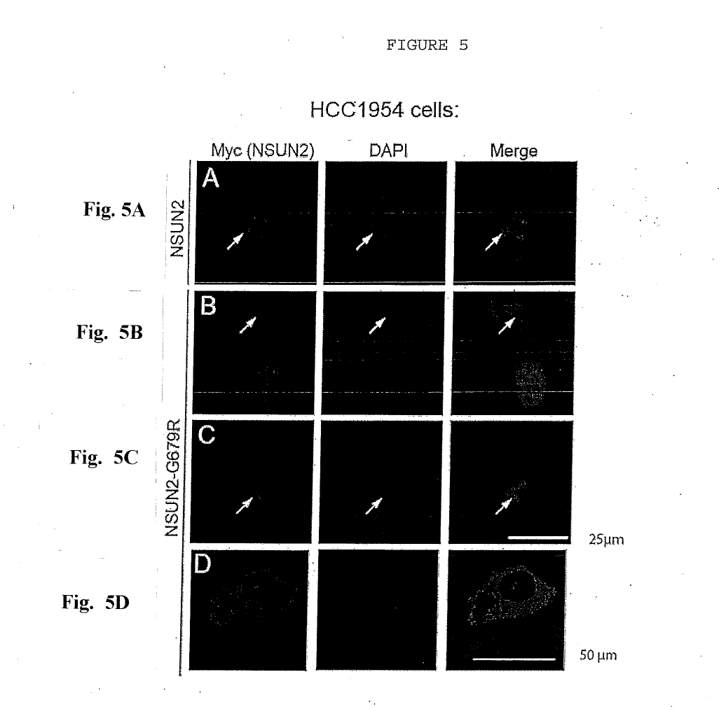

In these studies, a cDNA clone for NSUN2 in the vector pcDNA-Myc, site-directed mutagenesis was used to re-create the 2035 G>A/Gly679Arg mutation. Wild type (WT) and mutant constructs were transfected into the human breast cancer cell line HCC1954, and also into COST (monkey kidney) cells. Twenty-four hours later cells were stained with antibodies to the Myc epitope in order to detect transfected proteins. While the WT NSUN2 protein was detected in the nucleus and nucleolus of transfected HCC1954 cells (FIG. 5A), the Gly679Arg mutant failed to localize to the nucleoli in most transfected cells (FIGS. 5B and 5C). In rarer instances, the Gly679Arg mutant NSUN2 localized to the nucleoli and at the same time showed intense staining within the cytoplasm (FIG. 5D). Further, the mutant protein was largely excluded from the nucleoplasm in these cells, a staining pattern which was never observed with the transfected WT NSUN2. DAPI staining was used to show nuclear localization.

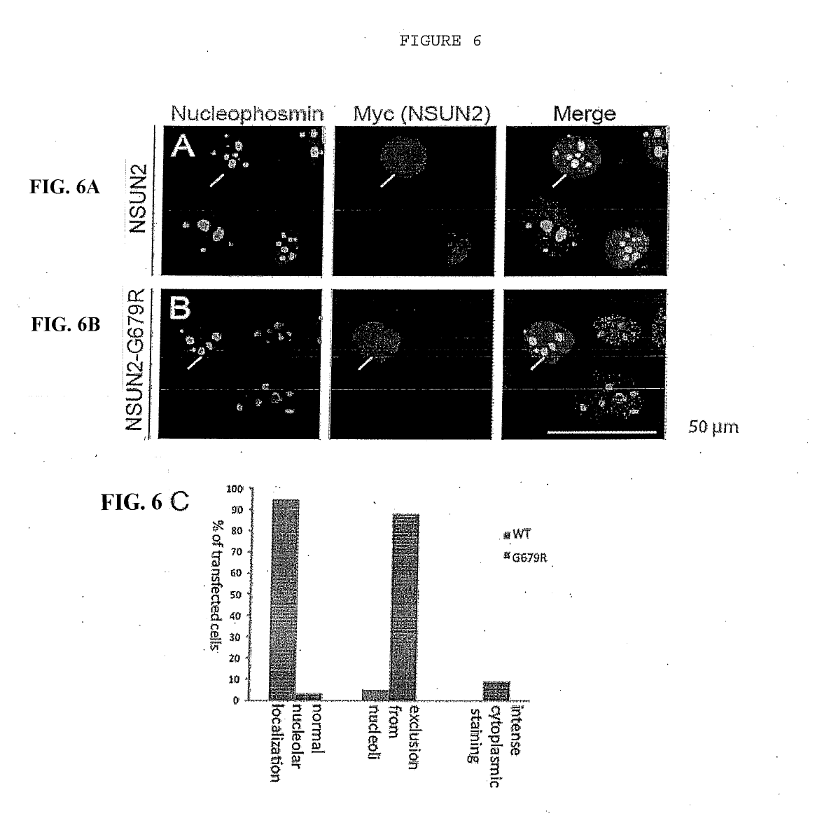

In addition, antibodies to the nucleolar marker protein, nucleophosmin (NPM1) were used to confirm co-localization in the nucleoli (FIGS. 6A and 6B), and quantification showed that 95% of cells transfected with WT NSUN2 exhibited normal nuclear localization, compared to only 3% of Gly679Arg mutant NSUN2. At the same time, 88% of cells with mutant NSUN2 exhibited exclusion of NSUN2 from the nucleoli, compared to 5% of WT cells, and 9% of mutant cells had intense cytoplasmic staining, compared to 0% for WT (FIG. 6C).

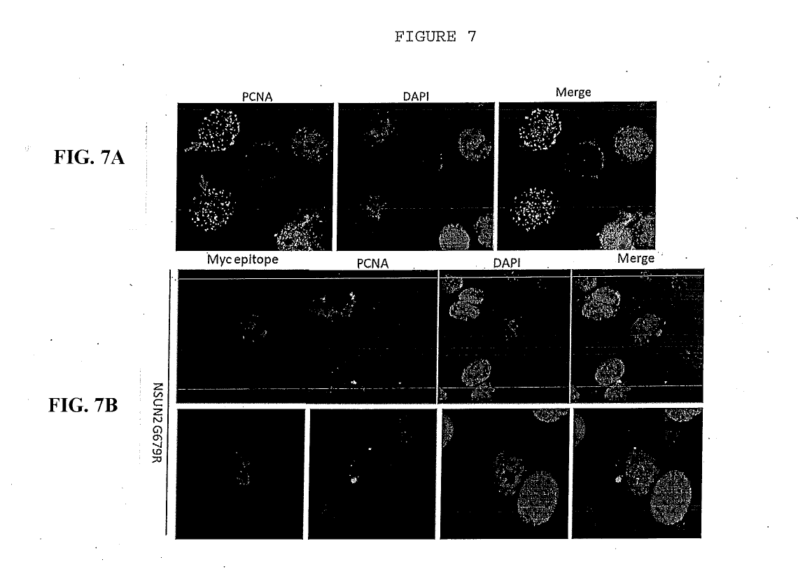

A co-staining with the proliferating cell nuclear antigen (PCNA) was used determine that the transfected cells displaying normal localization of the Gly679Arg mutant were not S-phase cells. When cells are fixed and extracted in methanol/acetone, S-phase cells are marked by a distinctively speckled staining of PCNA. However, as shown in FIGS. 7A and 7B, the speckled nuclear staining of PCNA was not detected in the few (3%) transfected cells which had a normal localisation of the Gly679Arg mutant (FIG. 7B).

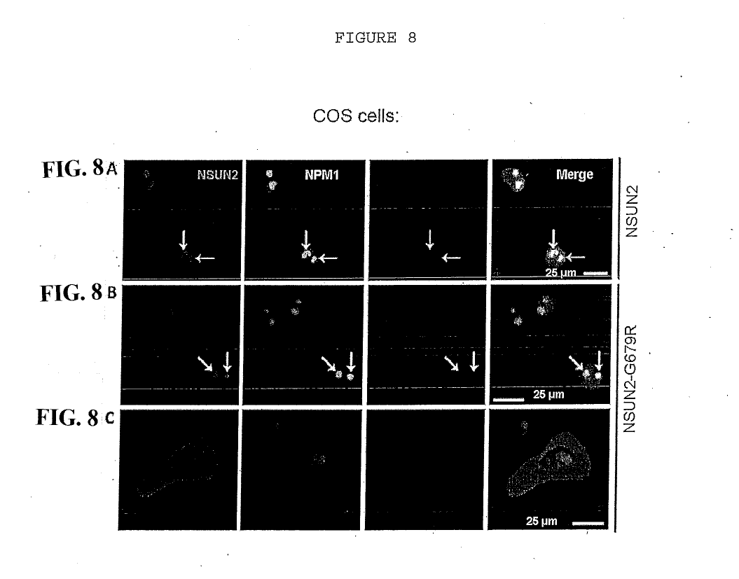

In transfected COST cells, overexpressed WT NSUN2 localized to the nucleus and nucleoli and was always excluded from the cytoplasm. In contrast, the Gly679Arg mutant, localised to the cytoplasm in most of these cells and was excluded from the nucleoli and nucleus. Thus, exclusion of the Gly679Arg mutant to the cytoplasm in these cells may be a species-specific effect. It was also observed that the mutant could still localise to the nucleoli in some cells during S-phase (see FIGS. 8A through 8C).

In parallel, cDNA constructs for WT and mutant were generated in pcDNA with GFP tag, and transfected into HeLa (human cervical cancer) cells, and into the human endothelial cell line EA.hy 926 (from umbilical vein). In transfected HeLa cells, after 24 hours plus a 2-3 hour incubation with colcemid (0.6 μg/ml of medium) a similar similar localization pattern to HCC1954 for WT and mutant was observed, with 34% GFP signal for mutant NSUN2 cells from the cytoplasm compared to 5% for WT, and exclusion of mutant NSUN2 from the nucleoli. Also, in EA.hy 926 cells WT NSUN2-GFP colocalized with the nucleophosmin 1 antibody (Santa Cruz) in the nucleoli, whereas the Gly679Arg mutant NSUN2-GFP remained in the nucleoplasm.

Accordingly, the present invention provides isolated polynucleotides and polypeptides encoded thereby useful as genetic markers for intellectual disability.

By “polynucleotide” it is meant to refer to any polymeric form of nucleotides and includes both sense and antisense strands of RNA, cDNA, genomic DNA, and synthetic forms and mixed polymers of the above. “Polynucleotide” as used herein is synonymous with “nucleic acid” and “nucleic acid molecule.” The term “polynucleotide” usually refers to a molecule of at least 10 bases in length, unless otherwise specified. The term includes single and double stranded forms of DNA. In addition, a polynucleotide may include either or both naturally occurring and modified nucleotides linked together by naturally occurring and/or non-naturally occurring nucleotide linkages.

In one embodiment of the present invention, the isolated polynucleotide comprises a mutant NSUN2 gene. The terms “mutant”, “mutated”, or “mutation” when applied to polynucleotides mean that nucleotides in the polynucleotide may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within the polynucleotides. In the present invention, the reference nucleic acid sequence is the wild type NSUN2 gene. Wild-type NSUN2 is disclosed in, for example, GENBANK sequence accession No. AK291144, SEQ ID NO:1 which is depicted herein in FIG. 9. The amino acid sequence encoded by wild-type NSUN2, SEQ ID NO:3, is depicted in FIG. 11. Alternative known nomenclature for the NSUN2 gene includes nol1/nop2/sun domain family, member 2; substrate of aim1/aurora kinase b (saki); myc-induced sun domain-containing protein (misu), trm4, and S. cerevisiae, homolog of trm4. In one embodiment of the present invention, the isolated polynucleotide comprises a mutation in exon 19 of the NSUN2 gene. In one embodiment of the present invention, the isolated polynucleotide of the present invention comprises the nucleic acid sequence of SEQ ID NO:2 depicted in FIG. 10 or a fragment thereof inclusive of the homozygous G>A base substitution at position 2035, a nucleic acid sequence encoding the amino acid sequence of SEQ ID NO:4 depicted in FIG. 12 or a fragment thereof, a nucleic acid sequence that selectively hybridizes under stringent conditions to the nucleic acid sequence of SEQ ID NO:2 depicted in FIG. 10 or a fragment thereof inclusive of the homozygous G>A base substitution at position 2035, or a nucleic acid sequence having at least 90% sequence identity, at least 95% sequence identity, at least 98% sequence identity, or at least 99% sequence identity, to the nucleic acid sequence of SEQ ID NO:2 depicted in FIG. 10 or a fragment thereof inclusive of the homozygous G>A base substitution at position 2035.

“Stringent hybridization conditions” and “stringent wash conditions” in the context of selective hybridization of polynucleotides of the present invention depends upon a number of different physical parameters. Important parameters include temperature of hybridization, base composition of the nucleic acids, salt concentration and length of the nucleic acid. Those of ordinary skill in the art understand how to vary these parameters to achieve a particular stringency of hybridization. In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See, e.g., Sambrook et al., Molecular Cloning: A Laboratory Manual, 2d ed., Cold Spring Harbor Laboratory Press (1989) and Sambrook et al., Molecular Cloning: A Laboratory Manual, 3d ed., Cold Spring Harbor Press (2001).

The Tₘ for a particular DNA-DNA hybrid can be estimated by the formula:

Tₘ=81.5° C.+16.6(log₁₀[Na⁺])+0.41(fraction G+C)−0.63(% formamide)−(600/l) where l is the length of the hybrid in base pairs.

The Tₘ for a particular RNA-RNA hybrid can be estimated by the formula:

Tₘ=79.8° C.+18.5(log₁₀[Na⁺])+0.58(fraction G+C)+11.8(fraction G+C)²−0.35(% formamide)−(820/l).

The Tₘ for a particular RNA-DNA hybrid can be estimated by the formula:

Tₘ=79.8° C.+18.5(log₁₀[Na⁺])+0.58(fraction G+C)+11.8(fraction G+C)²−0.50(% formamide)−(820/l).

In general, the Tₘ decreases by 1-1.5° C. for each 1% of mismatch between two nucleic acid sequences. Thus, one of ordinary skill in the art can alter hybridization and/or washing conditions to obtain sequences that have higher or lower degrees of sequence identity to the target nucleic acid. For instance, to obtain hybridizing nucleic acids that contain up to 10% mismatch from the target nucleic acid sequence, 10-15° C. would be subtracted from the calculated Tₘ of a perfectly matched hybrid, and then the hybridization and washing temperatures adjusted accordingly. Probe sequences may also hybridize specifically to duplex DNA under certain conditions to form triplex or other higher order DNA complexes. The preparation of such probes and suitable hybridization conditions are well known in the art.

Further, various methods to detect mutations in a nucleic acid sequence have been described and can be adapted routinely by those skilled in the art to detect additional mutations in the NSUN2 gene to those exemplified herein associated with intellectual disability. For example, but without limitation, mutations can be detected by primer extension, polymerase chain reaction (including long-range PCR) sequencing, single stranded conformation polymorphism, mismatch oligonucleotide mutation detection, mass spectroscopy, DNA microarray, HPLC, microarray, SNP PCR genotyping, or a combination thereof. Methods involving allele-specific probes for analyzing particular nucleotide sequences such as described by Saiki et al., Nature 324, 163-166 (1986) can also be used. Particular nucleic acid mutations can also be identified by hybridization to oligonucleotide arrays or subarrays such as described in WO 95/11995. Methods for determining the identity of the nucleotide present at a particular site that employs a specialized exonuclease-resistant nucleotide derivative can also be used. Solution-based methods for determining the identity of the nucleotide of a particular site such as described in U.S. Pat. No. 4,420,902 can also be used. Additional methods for detection of the nucleic acid genetic markers of the present invention include, but are not limited to Genetic Bit Analysis or GBA™, Oligonucleotide Ligation Assay or OLA, nucleic acid detection assays combining PCR and OLA, and primer-guided nucleotide incorporation procedures for assaying particular sites in DNA.

In one embodiment, the isolated polynucleotides of the present invention can be used in the development of hybridization probes to detect, characterize, and quantify hybridizing nucleic acids in, and isolate hybridizing nucleic acids from, both genomic and transcript-derived nucleic acid samples. Probes may be detectably labeled, particularly when used free in solution, or may be unlabeled, particularly when bound to a substrate, as in a microarray. Such probes can be used to detect and characterize alterations in the NSUN2 gene associated with intellectual disability.

The isolated polynucleotides of the present invention are also useful in the development of amplification primers.

In general, a probe or primer is at least 10 to 22 nucleotides in length. Primers and probes may also be longer in length. For instance, a probe or primer may be 25 nucleotides in length, or may be 30, 40 or 50 nucleotides in length. Nonlimiting examples of PCR primers of the present invention used for mutation screening of the NSUN2 gene are depicted in Table 2.

TABLE 2
NSUN2 Primers

| | | | Amplicon |
|---|---|---|---|
| Exon | Forward Primer(5′ to 3′) | Reverse Primer (5′ to 3′) | size (bp) |
| 1 | AAGTCCCGGGAGGTGTAGG | GGAAAGAGACGTCTACCCCG | 294 |
| | (SEQ ID NO: 5) | (SEQ ID NO: 6) | |
| 2 | GAAGTCCCGGGAGGTGTAG | GACCATTTCATTGGACCCTG | 628 |
| | (SEQ ID NO: 7) | (SEQ ID NO: 8) | |
| 3 | ACTCTCCATGCAAAAGTTGG | TGTTTGCAAAGTATACCCAAGC | 339 |
| | (SEQ ID NO: 9) | (SEQ ID NO: 10) | |
| 4 | AGCAGGGAGAACAACATTCG | TGTGTGATGTCCACCTACTGC | 335 |
| | (SEQ ID NO: 11) | (SEQ ID NO: 12) | |
| 5 | TGTTGCCTTTGAAACAATGC | ACGTACATATCACTCTCTATCCAAAG | 303 |
| | (SEQ ID NO: 13) | (SEQ ID NO: 14) | |
| 6 | TTTTGGGGCAGTGTAGAATC | ACGGTGGTAGGCAAGATGTC | 421 |
| | (SEQ ID NO: 15) | (SEQ ID NO: 16) | |
| 7 | TTCTCACTGGCTGACCTTTG | TGTGTCATATTCATGCACAACTTC | 375 |
| | (SEQ ID NO: 17) | (SEQ ID NO: 18) | |
| 8 | TGCAGTTGGTGAAAATGGAG | AAGATCGGATGCTCACTTGC | 300 |
| | (SEQ ID NO: 19) | (SEQ ID NO: 20) | |
| 9 | CTCTAACTTCATTTAGATCCCTGG | ACCTAACGGGACTGAACTGC | 408 |
| | (SEQ ID NO: 21) | (SEQ ID NO: 22) | |
| 10 | ACTTTCTGTCGACGGGTTTG | TCCGAAATTGATTCTAATTGGC | 357 |
| | (SEQ ID NO: 23) | (SEQ ID NO: 24) | |
| 11 | AGAGAATGGATACTCCTGCCC | ACCCCAACAAGACTCACCAG | 294 |
| | (SEQ ID NO: 25) | (SEQ ID NO: 26) | |
| 12 | GGCCATTCTATGAGTGAGGG | GCAAGAAGCTCCTCTTTCCTC | 329 |
| | (SEQ ID NO: 27) | (SEQ ID NO: 28) | |
| 13 | GATGGATTTTGTGAACGTGG | ACCAAGAAGTGGCTCTGGG | 365 |
| | (SEQ ID NO: 29) | (SEQ ID NO: 30) | |
| 14 | CCATGTTGCACAGAACTGAC | AGACAGAACTACATACAAAACAATGG | 297 |
| | (SEQ ID NO: 31) | (SEQ ID NO: 32) | |
| 15 | TTTTGGGGATTGAATTCTGG | TCACGGTCTGCTCCAAATG | 326 |
| | (SEQ ID NO: 33) | (SEQ ID NO: 34) | |
| 16 | GTGACGGTTGCTCCATGTG | CCTGAGCCACTCAGACCATC | 271 |
| | (SEQ ID NO: 35) | (SEQ ID NO: 36) | |
| 17 | AGCATAGCCCCTGAGAGCAG | ATAAATATGCCCCAACGCAC | 310 |
| | (SEQ ID NO: 37) | (SEQ ID NO: 38) | |
| 18 | GAATACTTAACACATCACTAGGTGCAG | GTTCCCACCTTCCCAAGTC | 229 |
| | (SEQ ID NO: 39) | (SEQ ID NO: 40) | |
| 19 | CTCTGGATTTGGTTTCAGACA | GGTATGGGTTTTCTGTGGTTT | 500 |
| | (SEQ ID NO: 41) | (SEQ ID NO: 42) | |

All amplicons amplified at 59 annealing temperature except for exons 1 and 2, for which a Q-solution (Qiagen, Mississauga, Ontario, CA) protocol was used.

Methods of performing nucleic acid hybridization using probes and methods of performing primer-directed amplification via, for example, PCR or RT-PCR are well known in the art.

PCR and hybridization methods may be used to identify and/or isolate polynucleotides of the present invention including allelic variants, homologous nucleic acid molecules and fragments. PCR and hybridization methods may also be used to identify, amplify and/or isolate polynucleotides of the present invention that encode homologous proteins. Nucleic acid primers as described herein can be used to prime amplification of polynucleotides of the present invention, using transcript-derived or genomic DNA as template.

The present invention also provides polypeptides as genetic markers of intellectual disability. The polypeptide is a mutant polypeptide as compared to the protein encoded by wild type NSUN2 gene. The term “mutant”, “mutated” or “mutation” when referring to a polypeptide of the present invention relates to an amino acid sequence containing substitutions, insertions or deletions of one or more amino acids compared to the amino acid sequence encoded by NSUN2 gene. In one embodiment, the isolated polypeptide of the present invention comprises an amino acid sequence mutated as compared to the amino acid sequence of SEQ ID NO:3 depicted in FIG. 11. In one embodiment of the present invention, the isolated polypeptide of the present invention comprises the amino acid sequence of SEQ ID NO:4 depicted in FIG. 12 or a fragment thereof inclusive of the missense mutation Gly679Arg.

The present invention also provides methods for screening an individual for a genetic marker associated with intellectual disability. In this method, a sample obtained from the individual is assayed for the presence of a genetic marker of the present invention. Presence of the genetic marker in the individual indicates that the individual has a gene sequence associated with intellectual disability. In one embodiment of this method, a sample comprising DNA or RNA is obtained from the individual and the sample is assayed for mutations in the NSUN2 gene indicative of intellectual disability. In another embodiment, a sample comprising proteins is obtained from the individual and the sample is tested for the presence of a mutant polypeptide as compared to the protein encoded by wild type NSUN2 gene. Presence of mutant polypeptide may be detected as a change in subcellular localization, level, activity and/or structure as compared to protein encoded by wild type NSUN2 gene.

The present invention also provides methods for genetically diagnosing intellectual disability in an individual by detecting in the individual a genetic marker of the present invention. In one embodiment of this method, a sample comprising DNA or RNA is obtained from the individual and the sample is assayed for mutations in the NSUN2 gene indicative of intellectual disability. In another embodiment, a sample comprising proteins is obtained from the individual and the sample is tested for the presence of a mutant polypeptide as compared to the protein encoded by wild type NSUN2 gene. Presence of mutant polypeptide may be detected as a change in subcellular localization, level, activity and/or structure as compared to protein encoded by wild type NSUN2 gene.

The present invention also provides methods for identifying individuals predisposed genetically to offspring suffering from intellectual disability by detection of these genetic markers. As intellectual disability is a recessive trait, carriers are heterozygous for a mutant NSUN2 gene. Mating of two carriers results in a one in four chance that the offspring will by homozygous for a mutant NSUN2 gene and intellectually disabled. Carrier status is detected by obtaining a sample comprising DNA from an individual and sequencing the DNA in the sample.

Samples obtained from an individual which can be analyzed in accordance with these methods may comprise any tissue or biological fluid sample from which DNA, RNA and/or proteins can be obtained. Examples include, but are in no way limited to, blood, plasma, serum, hair follicle cells, skin cells, cheek cells, saliva cells, tissue biopsy, and the like. In one embodiment, the sample is blood or serum.

Various methods and reagents for detection of nucleic acid sequences have been described and can be adapted routinely by those skilled in the art for detection of the genetic markers described herein. Examples include, but are in no way limited to, hybridization assays, nucleotide sequencing, PCR, and combinations thereof. mRNA expression can also be measured by, for example, Northern blot, quantitative or qualitative reverse transcriptase PCR (RT-PCR), microarray, dot or slot blots and in situ hybridization.

Various methods and reagents for detection of proteins have also been described and can be adapted routinely by those skilled in the art for detection of the genetic markers described herein. Examples of methods for determining altered levels of polypeptide expression include, but are not limited to, radioimmunoassays, competitive-binding assays, ELISA, Western blot, FACS, immunohistochemistry, immunoprecipitation, proteomic approaches: two-dimensional gel electrophoresis (2D electrophoresis) and non-gel-based approaches such as mass spectrometry or protein interaction profiling. Alterations in the structure of a polypeptide encoded by a mutant NSUN2 gene may be determined by any method known in the art, including, but not limited to use of antibodies that specifically recognize a mutated residue, two-dimensional polyacrylamide gel electrophoresis (2D PAGE) and/or chemical analysis of amino acid residues of the protein.

Identification of mutations in the NSUN2 gene and/or the polypeptide encoded thereby is expected to lead to identification of proteins with a unique structure, protein subcellular localization, biochemical processing and/or function. This information can be used to directly or indirectly to facilitate the generation of therapeutics and additional diagnostics for intellectual disability. Specifically, the polynucleotides and polypeptides of the present invention may enable the production of antibodies or compounds directed against the novel region for use as a therapeutic or diagnostic. Alternatively, the polynucleotides and polypeptides of the present invention may alter the biochemical or biological properties of the encoded protein in such a way as to enable the generation of improved or different therapeutics targeting this protein. One may determine whether polypeptides of the present invention are functional by methods known in the art. For example, residues that are tolerant of change while retaining function can be identified by altering the polypeptide at known residues using methods known in the art, such as alanine scanning mutagenesis (Cunningham et al. 1989 Science 244(4908):1081-5), transposon linker scanning mutagenesis (Chen et al. Gene 2001 263(1-2): 39-48); combinations of homolog- and alanine-scanning mutagenesis (Jin et al. J. Mol. Biol. 1992 226(3):851-65) and/or combinatorial alanine scanning (Weiss et al. Proc. Natl. Acad. Sci USA 2000 97(16):8950-4), followed by functional assay.

The present invention also provides for kits for screening individuals for a genetic marker of intellectual disability, genetically diagnosing intellectual disability and/or identifying individuals predisposed genetically to offspring suffering from intellectual disability. Any of the means described herein for identification of a mutation in NSUN2 may be comprised in a kit. In a non-limiting example, a probe or primer, control nucleic acid (including wildtype NSUN2), and amplification reagents may be comprised in a kit in suitable container means. The components of the kits may be packaged either in aqueous media or, for example, in lyophilized form. The kits may further comprise at least one vial, test tube, flask, bottle, syringe and/or other container means, into which a component may be placed. Where there is more than one component in the kit, the kit may further comprise additional containers into which the additional components may be separately placed. Kits of the present invention will also typically comprise written instructions for their use. Kits of the present invention may also comprise a means for containing the components of the kit in close confinement for commercial sale and/or storage.

The following nonlimiting examples are provided to further illustrate the present invention.

### EXAMPLES

### Example 1

### Patients

The family ascertained in this study is from a farming community in the district of Khairpur, within the province of Sindh in Pakistan. The pedigree structure indicates a high degree of consanguinity (see FIG. 1), with first-cousin marriages for the parents of all the affected individuals, and the presence of intellectual disability among the male and female offspring in three branches of the family suggesting an autosomal recessive pattern of inheritance. Appropriate informed consent was obtained for all participants in the study. The patients were assessed by a consultant in psychiatry specializing in learning disability/mental retardation, who trained both in Pakistan and the United Kingdom, and is fluent in the Punjabi and Urdu languages. The patients as well as a number of relatives were assessed. Information was obtained about early development, schooling and academic achievement, current level of social functioning and adjustment and support and help required for different activities of daily living. Based on this information a judgment was made about the level of impairment the patients suffered using the relatives as reference for normal functioning. A consensus diagnosis was reached that all the affected individuals had intellectual disability of mild to moderate degree.

Neurological assessment was performed by a consultant neurologist. Clinical examination of affected individuals revealed that motor development was delayed, occipito-frontal circumferences were within the normal range and facial appearances were normal. There were no dysmorphic features, no hepatosplenomegaly, no heart murmur, and no skin abnormalities. Computed tomography of the brain was performed for two affected individuals, which was generally normal. Affected individuals had normal ventricles and cerebral volume. Gray and white matter differentiation was preserved, and posterior fossa was unremarkable.

Photographs of all affected individuals were assessed for dysmorphic features by an experienced clinical geneticist. No unusual facial features were apparent.

### Example 2

### Clinical Description of Patients

Case 1: A 13 year old girl from Khairpur district, Sindh province (Pakistan) presented with a history of developmental delay, poor cognitive development and aggressive behavior. Her antenatal history is unclear but the mother did not recall any undue complications no ultrasounds were done antenatally. She was born at full term at home via spontaneous vertex delivery. There was apparently no complication noted during or after the time of delivery. Although the mother did not remember the exact milestones, she walked late at around 4 years of age, her speech developed a few years after she started to walk and even at 13 years she could not speak clearly and does not recall her name when asked. She was toilet trained and was able to feed herself, dress and undress by herself, however she was not able to help with daily household chores. She had no history of seizures or loss of consciousness.

Her parents had a consanguineous marriage and were first cousins. She had 6 siblings, 3 males and 3 females. Two of her female siblings had similar issues of delayed and poor cognition and development (see below). There was a strong family history, with one of her male cousins (maternal uncle's son) who also had intellectual disability. The remaining siblings were normal. There was no history of any abortions or still births in the mother.

On examination, her weight was 26.3 kg (<5% tile) and height was 136 cms (<5% centile), and head circumference was 49 cm. Facial features were described as long facies, long pointed nose, pointed chin and wide mouth. She had mild webbing of the neck. She was at Tanner stage 4 and had started her menstruation very recently.

Neurological exam showed that she was alert, was oriented in space and person, and a little shy in her demeanor. Her speech was dysarthric. Her gait was broad based, and she had bilateral pes cavus with some equinus position of both her feet. Her Achilles was tight bilaterally. Her tone was increased in all her limbs and reflexes were brisk. Planters were equivocal. Her cranial nerves were intact. She had normal pupillary response to light, her eye movements were equal in all directions and her face was symmetrical. She had a good gag reflex. Her fundoscopy showed a normal disc. Coordination and sensory exam was grossly normal. Her cardiovascular, respiratory and abdominal exam was normal.

Case 2: A 14 year old girl, who is the older sister of Case 1, was also developmentally delayed and had poor cognitive development. She is the first born and apparently the mother did not notice anything unusual during the antenatal period. She was born full term at home, but cried a little late. She was not hospitalized after birth, nor was there any need for giving oxygen to the baby. There was no jaundice during the early neonatal period. Her developmental milestones were also significantly delayed. She walked at around 5 years and spoke after 5 years of age. She had no history of seizures or loss of consciousness, however after 3 years of age she developed deviation of her right eye which happened after fever. She is able to perform her daily living activities at home, and helps in the household chores. She was never sent to any kind of school.

On examination, her weight was 32 kg (<5th centile) and her height was 152 cm (<5^{th} centile). Her head circumference was 50 cms. She was at Tanner stage 3, and according to the mother she had not started her menstruation as yet. She had lateral strabismus of her right eye, had a long facies, long pointed nose and pointed chin and webbing of her neck. Her fingers were tapering, there was no hyper extensibility noted. Her left foot had pes cavus and the Achilles on left foot was tight. The right foot was normal. Her overall peripheral tone was increased, power was grade 5 and reflexes were brisk. Planters were equivocal. Her gait was broad based and face was symmetrical. Speech was limited to a few words and dysathria was present. Sensory examination and fundoscopy was difficult as she was not cooperative. Rest of her systemic examination was normal.

Case 3: A 6 year old girl, the youngest of all her siblings was also developmentally delayed with no speech development. She had started walking a year ago. On examination she had pectus excavatum, webbing of the neck, brachycephaly with a head circumference of 46 cms (<5^{th} centile). She had partial syndactyly of her 1^{st} and 2^{nd} toe bilaterally. No pes cavus was seen. Her gait was normal, tone was normal, and reflexes were brisk with unsustained clonus. Eye examination showed alternating esotropia and fine horizontal nystagmus, fundus examination could not be done. The rest of her cranial nerve examination was grossly normal. Cardiovascular respiratory and abdominal exam were unremarkable

### Example 3

### Sample Collection and DNA Extraction

Blood samples were collected from a total of eight family members, three of which were affected. Genomic DNA was extracted from peripheral blood leukocytes by standard methods such as described by Lahiri et al. in Nucleic Acids Res. 1991 19(19):5444.

### Example 4

### Single Nucleotide Polymorphism (SNP) Homozygosity Mapping

DNA samples of three affected and one unaffected members of the family were analyzed using the Affymetrix GENECHIP Mapping 500K array (Affymetrix, Inc. Santa Clara, Calif.). These arrays allow analysis of approximately 500,000 single nucleotide polymorphisms with a median physical distance of 2.5 kb and an average physical distance of 5.8 Kb between single nucleotide polymorphisms. The average heterozygosity of these single nucleotide polymorphisms is 0.30. In these experiments, the NspI chip from the GENECHIP Mapping 500K set was used, which allowed genotyping of approximately 260,000 single nucleotide polymorphisms in the patient DNAs. Sample processing, labeling and hybridization were performed in accordance with the manufacturer's instructions (Affymetrix Mapping 500K Assay Manual). The arrays were scanned with a GENECHIP (Affymetrix Scanner and the data was processed using GENECHIP Operating Software (GCOS) and GENECHIP Genotyping Analysis Software (GTYPE) Software (ver. 3.0.2) to generate SNP allele calls.

Using Affymetrix 5.0 SNP microarrays, 2.565 Mb region was identified on 5p15.32-p15.31 with a continuous run of 798 SNPs homozygous common among all affecteds in the family. The boundaries of the homozygosity-by-descent (HBD) region was set in this family by the flanking SNPs rs2259 and rs2914296, defining a ˜5 Mb region. This region overlaps with a locus previously identified as harboring a gene for NS-ARMR, and has been designated MRT5, however no causative gene has yet been reported for this locus (Najmabadi et al. Hum Genet 2007; 121:43-48). The MRT5 locus was defined by SNP markers rs1824938 (5.092 Mb) and rs60701 (10.734 Mb). Thus, the common region shared between our Pakistani family and the Iranian family was from rs1824938 (5.092 Mb) to rs2914296 (7.657 Mb)—a 2.565 critical region.

### Example 5

### Copy Number Analysis

Copy Number variations that include deletions, and duplication events were inferred by comparative analysis of hybridization intensities using dChip analyzer (Li C, Wong W H (2003). DNA-Chip Analyzer (dChip). In The analysis of gene expression data: methods and software. Edited by G Parmigiani, E S Garrett, R Irizarry and S L Zeger. Springer; Zhao et al. Cancer Res 2004 64:3060-3071; and Zhao et al. Cancer Res 2005 65:5561-5570 2005). After normalization, CNAG (Nannya et al. Cancer Res 2005 65:6071-6079), which employs a Hidden Markov Model, was used to infer the DNA copy number from the raw signal data. This algorithm uses pairwise data comparisons rather than comparison across all samples (as in dChip), and has a better signal to noise ratio. Both algorithms were used and compared.

### Example 6

### DNA Analysis with Microsatellite Markers and Linkage Analysis

Six microsatellite markers (Table 1) across the 5p region were PCR amplified using standard protocols such as described in Molecular Cloning: A Laboratory Manual (Third Edition) By Joseph Sambrook and David Russell, 2001, Cold Spring Harbor Laboratory Press, NY and were electrophoresed on an ABI 3730xl DNA analyzer. The genotypes were called using Genemapper software (Applied Biosystems of Life Technologies, Carlsbad, Calif.) and linkage analysis was performed using MLINK software, part of the LINKAGE software package described by Lathrop et al. in. Am J Hum Genet. 1985 37(3):482-98.

### Example 7

### Mutation Screening

Proband DNA was screened for mutations by PCR followed by ABI BigDye™ (Applied Biosystems of Life Technologies, Carlsbad, Calif.) sequencing for each coding exon.

What is claimed is:

1. A probe which detects a genetic marker for intellectual disability comprising a mutant NSUN2 gene or a mutant polypeptide encoded thereby, said mutant comprising a point mutation in exon 19 of the NSUN2 gene resulting in a missense mutation in the polypeptide encoded thereby, wherein said probe is detectably labeled or bound to a substrate.

2. The probe of claim 1 wherein said mutant comprises a substitution of G at nucleotide position 2035 from the translation start site in SEQ ID NO:1 or substitution of Gly at position 679 of the amino acid sequence of SEQ ID NO:3.

3. The probe of claim 2 wherein said mutant comprises a G to A substitution corresponding to nucleotide position 2035 from the translation start site in SEQ ID NO:1 or a Gly to Arg substitution at position 679 of the amino acid sequence of SEQ ID NO:3.

4. The probe of claim 1 which is at least 10 to 50 nucleotides in length.

5. A primer pair which amplifies a genetic marker for intellectual disability comprising a mutant NSUN2 gene or a mutant polypeptide encoded thereby, said mutant comprising a point mutation in exon 19 of the NSUN2 gene resulting in a missense mutation in the polypeptide encoded thereby.

6. The primer pair of claim 5 wherein the mutant comprises a substitution of G at nucleotide position 2035 from the translation start site in SEQ ID NO:1 or substitution of Gly at position 679 of the amino acid sequence of SEQ ID NO:3.

7. The primer pair of claim 6 wherein said mutant comprises a G to A substitution corresponding to nucleotide position 2035 from the translation start site in SEQ ID NO:1 or a Gly to Arg substitution at position 679 of the amino acid sequence of SEQ ID NO:3

8. The primer pair of claim 5 wherein the primers are at least 10 to 50 nucleotides in length.

9. The primer pair of claim 5 comprising an NSUN2 primer pair selected from the group consisting of SEQ ID NO:5 and SEQ ID NO:6, SEQ ID NO:7 and SEQ ID NO:8, SEQ ID NO:9 and SEQ ID NO:10, SEQ ID NO:11 and SEQ ID NO:12, SEQ ID NO:13 and SEQ ID NO:14, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:23 and SEQ ID NO:24, SEQ ID NO:25 and SEQ ID NO:26, SEQ ID NO:27 and SEQ ID NO:28, SEQ ID NO:29 and SEQ ID NO:30, SEQ ID NO:31 and SEQ ID NO:32, SEQ ID NO:33 and SEQ ID NO:34, SEQ ID NO:35 and SEQ ID NO:36, SEQ ID NO:37 and SEQ ID NO:38, SEQ ID NO:39 and SEQ ID NO:40, and SEQ ID NO:41 and SEQ ID NO:42.

10. A synthetic polynucleotide comprising:
(a) a nucleic acid sequence of SEQ ID NO:2;
(b) a nucleic acid sequence encoding the amino acid sequence of SEQ ID NO:4;
(c) a nucleic acid sequence that selectively hybridizes to the nucleic acid sequence of (a) or (b); or
(d) a nucleic acid sequence having at least 90% sequence identity to the nucleic acid molecule of (a).

11. The synthetic polynucleotide of claim 10 comprising cDNA.

12. An isolated polypeptide comprising the amino acid sequence of SEQ ID NO:4.

13. An antibody produced against a polypeptide encoded by a mutant NSUN2 gene or a fragment thereof, said antibody specifically recognizing the mutant polypeptide.

14. A method of screening an individual for a genetic marker associated with intellectual disability, said method comprising detecting in a sample obtained from the individual the genetic marker with the probe of claim 1 wherein the presence of the genetic marker in the individual indicates that the individual has a gene sequence associated with intellectual disability.

15. A method of genetically diagnosing intellectual disability in an individual, said method comprising detecting in a sample obtained from the individual the genetic marker with the probe of claim 1, wherein the presence of the genetic marker, is indicative of the individual being intellectually disabled.

16. A method for identifying an individual predisposed genetically to offspring suffering from intellectual disability, said method comprising detecting in a sample obtained from the individual the genetic marker with the probe of claim 1, wherein the presence of the genetic marker is indicative of the individual being predisposed genetically to offspring suffering from intellectual disability.

17. A kit to screen an individual for a genetic marker associated with intellectual disability, identify genetically individuals suffering from intellectual disability, and/or identify individuals predisposed genetically to offspring suffering from intellectual disability, said kit comprising a means for detecting a genetic marker for intellectual disability comprising a mutant NSUN2 gene or a mutant polypeptide encoded thereby, said mutant comprising a point mutation in exon 19 of the NSUN2 gene resulting in a missense mutation in the polypeptide encoded thereby.

18. The kit of claim 17 wherein said means comprises a probe which detects the genetic marker for intellectual disability comprising the mutant NSUN2 gene or a mutant polypeptide encoded thereby, wherein said probe is detectably labeled or bound to a substrate.

19. The kit of claim 17 wherein said means comprises an antibody which specifically recognizes the genetic marker for intellectual disability comprising the mutant NSUN2 gene or a mutant polypeptide encoded thereby.


SEQUENCE LISTING (42 sequences)

SEQ ID NO: 1 - DNA, 2634 bp, Homo sapien
gggggctagagttctggccgtggcgggccggtttctgcgtgctgcgtgcgcggccgcgtgggctatggggcggcggtcgcggggtcggcggctccagcaacagcagcggccggaagacgcggaggatggcgccgagggtggtggaaagcgcggcgaggcgggctgggaaggaggctaccccgagatcgtcaaggagaacaagctgttcgagcactactaccaggagctcaagatcgtgcccgagggcgagtggggccagttcatggacgctctcagggagccgctcccggccactttaagaattactggttacaaaagccacgcaaaagagattctccattgcttaaagaacaaatattttaaggaattggaggacctggaggtggacggtcagaaagttgaagttccacagccactgagttggtatcctgaagaacttgcctggcacacaaatttaagtcgaaaaatcttgagaaaatcgccacacttggaaaagtttcatcagtttctagttagtgaaacagaatctggaaatattagtcgtcaagaagctgttagcatgatcccaccactgctcctcaacgtgcggcctcatcataagatcttagatatgtgtgcagcacctggctcaaagaccacacagttaattgaaatgctacatgccgacatgaatgtcccctttccagagggatttgttattgcgaatgatgtggacaacaagcgctgctacctgctcgtccatcaagccaagaggctgagcagcccctgcatcatggtggtcaaccatgatgcctccagcatacccaggctccagatagatgtggacggcaggaaagagatcctcttctatgatcgaattttatgtgatgtcccttgcagtggagacggcactatgagaaaaaacattgatgtttggaaaaagtggaccaccttaaatagcttgcagctacatggcttacagctgcggattgcaacacgcggggctgaacagctggctgaaggtggaaggatggtgtattccacgtgttcactaaaccctattggggatgaagcagtcatagcatctttactggaaaaaagtgaaggtgctttggagcttgctgatgtgtctaatgaactgccagggctgaagtggatgcctggaatcacacagtggaaggtaatgacgaaagatgggcagtggtttacagactgggacgctgttcctcacagcagacacacccagatccgacctaccatgttccctccgaaggacccagaaaagctgcaggccatgcacctggagcgatgccttaggatattaccccatcatcagaatactggagggttttttgtggcagtattggtgaaaaaatcttcaatgccgtggaataaacgtcagccaaagcttcagggtaaatctgcagagaccagagaaagcacacagctgagccctgcagatctcacagaagggaaacccacagatccctctaagctggaaagtccgtcattcacaggaactggtgacacagaaatagctcatgcaactgaggatttagagaataatggcagtaagaaagatggcgtgtgtggtcctcctccatcaaagaaaatgaagttatttggatttaaagaagatccatttgtatttattcctgaagatgacccattatttccacctattgagaaattttatgctttggatccttcattcccaaggatgaatttgttaactcggactacagaagggaagaaaaggcagctctacatggtttctaaggagttgcggaatgtgctgctgaataacagtgagaagatgaaggttattaacacggggatcaaagtctggtgtagaaataacagcggtgaagagtttgactgtgctttccggctggcacaggagggaatatatacattgtatccatttattaactcaagaattattactgtatcaatggaagatgttaagatactgttgacccaggaaaatcccttttttagaaaactcagcagtgagacctacagtcaagcaaaggacctggcaaagggaagcatcgtgctgaagtatgaaccagattctgcgaatccagacgctctgcagtgtcccatcgtcttatgcggatggcggggaaaggcctccattcgaacttttgtgcccaagaatgaacggcttcattatctcaggatgatggggctggaggtattgggagaaaagaagaaggaaggggttatcctcacaaatgagagtgcagccagcaccggacagccagacaatgacgtgactgagggacagagagcaggagagcccaacagcccagatgcagaagaggccaacagtccagacgtgacagcaggctgtgacccggcgggggtccatccaccccggtgagcaggcccaaggcagcgggggcccacacccctcacacgcaaaactggcttcttctggtcactggtgtctgaaaccaaatccagagcagcctgtggcctgtaaagcatatatttctaatgactgcagactggtgggatcataggagccttctgaatgaccaggactgctttctttggagctgatgaaaatgtactcttttagcgtgttagaaatcacttgttttattttgtttctttggccaagctgggtctagtgtttcttttgct

SEQ ID NO: 2 - DNA, 2570 bp, Homo sapien
atggggcggcggtcgcggggtcggcggctccagcaacagcagcggccggaagacgcggaggatggcgccgagggtggtggaaagcgcggcgaggcgggctgggaaggaggctaccccgagatcgtcaaggagaacaagctgttcgagcactactaccaggagctcaagatcgtgcccgagggcgagtggggccagttcatggacgctctcagggagccgctcccggccactttaagaattactggttacaaaagccacgcaaaagagattctccattgcttaaagaacaaatattttaaggaattggaggacctggaggtggacggtcagaaagttgaagttccacagccactgagttggtatcctgaagaacttgcctggcacacaaatttaagtcgaaaaatcttgagaaaatcgccacacttggaaaagtttcatcagtttctagttagtgaaacagaatctggaaatattagtcgtcaagaagctgttagcatgatcccaccactgctcctcaacgtgcggcctcatcataagatcttagatatgtgtgcagcacctggctcaaagaccacacagttaattgaaatgctacatgccgacatgaatgtcccctttccagagggatttgttattgcgaatgatgtggacaacaagcgctgctacctgctcgtccatcaagccaagaggctgagcagcccctgcatcatggtggtcaaccatgatgcctccagcatacccaggctccagatagatgtggacggcaggaaagagatcctcttctatgatcgaattttatgtgatgtcccttgcagtggagacggcactatgagaaaaaacattgatgtttggaaaaagtggaccaccttaaatagcttgcagctacatggcttacagctgcggattgcaacacgcggggctgaacagctggctgaaggtggaaggatggtgtattccacgtgttcactaaaccctattggggatgaagcagtcatagcatctttactggaaaaaagtgaaggtgctttggagcttgctgatgtgtctaatgaactgccagggctgaagtggatgcctggaatcacacagtggaaggtaatgacgaaagatgggcagtggtttacagactgggacgctgttcctcacagcagacacacccagatccgacctaccatgttccctccgaaggacccagaaaagctgcaggccatgcacctggagcgatgccttaggatattaccccatcatcagaatactggagggttttttgtggcagtattggtgaaaaaatcttcaatgccgtggaataaacgtcagccaaagcttcagggtaaatctgcagagaccagagaaagcacacagctgagccctgcagatctcacagaagggaaacccacagatccctctaagctggaaagtccgtcattcacaggaactggtgacacagaaatagctcatgcaactgaggatttagagaataatggcagtaagaaagatggcgtgtgtggtcctcctccatcaaagaaaatgaagttatttggatttaaagaagatccatttgtatttattcctgaagatgacccattatttccacctattgagaaattttatgctttggatccttcattcccaaggatgaatttgttaactcggactacagaagggaagaaaaggcagctctacatggtttctaaggagttgcggaatgtgctgctgaataacagtgagaagatgaaggttattaacacggggatcaaagtctggtgtagaaataacagcggtgaagagtttgactgtgctttccggctggcacaggagggaatatatacattgtatccatttattaactcaagaattattactgtatcaatggaagatgttaagatactgttgacccaggaaaatcccttttttagaaaactcagcagtgagacctacagtcaagcaaaggacctggcaaagggaagcatcgtgctgaagtatgaaccagattctgcgaatccagacgctctgcagtgtcccatcgtcttatgcagatggcggggaaaggcctccattcgaacttttgtgcccaagaatgaacggcttcattatctcaggatgatggggctggaggtattgggagaaaagaagaaggaaggggttatcctcacaaatgagagtgcagccagcaccggacagccagacaatgacgtgactgagggacagagagcaggagagcccaacagcccagatgcagaagaggccaacagtccagacgtgacagcaggctgtgacccggcgggggtccatccaccccggtgagcaggcccaaggcagcgggggcccacacccctcacacgcaaaactggcttcttctggtcactggtgtctgaaaccaaatccagagcagcctgtggcctgtaaagcatatatttctaatgactgcagactggtgggatcataggagccttctgaatgaccaggactgctttctttggagctgatgaaaatgtactcttttagcgtgttagaaatcacttgttttattttgtttctttggccaagctgggtctagtgtttcttttgct

SEQ ID NO: 3 - protein, 767 aa, Homo sapien [sequence omitted]

SEQ ID NO: 4 - protein, 767 aa, Homo sapien
MGRRSRGRRLQQQQRPEDAEDGAEGGGKRGEAGWEGGYPEIVKENKLFEHYYQELKIVPEGEWGQFMDALREPLPATLRITGYKSHAKEILHCLKNKYFKELEDLEVDGQKVEVPQPLSWYPEELAWHTNLSRKILRKSPHLEKFHQFLVSETESGNISRQEAVSMIPPLLLNVRPHHKILDMCAAPGSKTTQLIEMLHADMNVPFPEGFVIANDVDNKRCYLLVHQAKRLSSPCIMVVNHDASSIPRLQIDVDGRKEILFYDRILCDVPCSGDGTMRKNIDVWKKWTTLNSLQLHGLQLRIATRGAEQLAEGGRMVYSTCSLNPIGDEAVIASLLEKSEGALELADVSNELPGLKWMPGITQWKVMTKDGQWFTDWDAVPHSRHTQIRPTMFPPKDPEKLQAMHLERCLRILPHHQNTGGFFVAVLVKKSSMPWNKRQPKLQGKSAETRESTQLSPADLTEGKPTDPSKLESPSFTGTGDTEIAHATEDLENNGSKKDGVCGPPPSKKMKLFGFKEDPFVFIPEDDPLFPPIEKFYALDPSFPRMNLLTRTTEGKKRQLYMVSKELRNVLLNNSEKMKVINTGIKVWCRNNSGEEFDCAFRLAQEGIYTLYPFINSRIITVSMEDVKILLTQENPFFRKLSSETYSQAKDLAKGSIVLKYEPDSANPDALQCPIVLCRWRGKASIRTFVPKNERLHYLRMMGLEVLGEKKKEGVILTNESAASTGQPDNDVTEGQRAGEPNSPDAEEANSPDVTAGCDPAGVHPPR

SEQ ID NO: 5 - DNA, 19 bp, Artificial sequence
  Synthetic
aagtcccgggaggtgtagg

SEQ ID NO: 6 - DNA, 20 bp, Artificial sequence
  Synthetic
ggaaagagacgtctaccccg

SEQ ID NO: 7 - DNA, 19 bp, Artificial sequence
  Synthetic
gaagtcccgggaggtgtag

SEQ ID NO: 8 - DNA, 20 bp, Artificial sequence
  Synthetic
gaccatttcattggaccctg

SEQ ID NO: 9 - DNA, 20 bp, Artificial sequence
  Synthetic
actctccatgcaaaagttgg

SEQ ID NO: 10 - DNA, 22 bp, Artificial sequence
  Synthetic
tgtttgcaaagtatacccaagc

SEQ ID NO: 11 - DNA, 20 bp, Artificial sequence
  Synthetic
agcagggagaacaacattcg

SEQ ID NO: 12 - DNA, 21 bp, Artificial sequence
  Synthetic
tgtgtgatgtccacctactgc

SEQ ID NO: 13 - DNA, 20 bp, Artificial sequence
  Synthetic
tgttgcctttgaaacaatgc

SEQ ID NO: 14 - DNA, 26 bp, Artificial sequence
  Synthetic
acgtacatatcactctctatccaaag

SEQ ID NO: 15 - DNA, 20 bp, Artificial sequence
  Synthetic
ttttggggcagtgtagaatc

SEQ ID NO: 16 - DNA, 20 bp, Artificial sequence
  Synthetic
acggtggtaggcaagatgtc

SEQ ID NO: 17 - DNA, 20 bp, Artificial sequence
  Synthetic
ttctcactggctgacctttg

SEQ ID NO: 18 - DNA, 24 bp, Artificial sequence
  Synthetic
tgtgtcatattcatgcacaacttc

SEQ ID NO: 19 - DNA, 20 bp, Artificial sequence
  Synthetic
tgcagttggtgaaaatggag

SEQ ID NO: 20 - DNA, 20 bp, Artificial sequence
  Synthetic
aagatcggatgctcacttgc

SEQ ID NO: 21 - DNA, 24 bp, Artificial sequence
  Synthetic
ctctaacttcatttagatccctgg

SEQ ID NO: 22 - DNA, 20 bp, Artificial sequence
  Synthetic
acctaacgggactgaactgc

SEQ ID NO: 23 - DNA, 20 bp, Artificial sequence
  Synthetic
actttctgtcgacgggtttg

SEQ ID NO: 24 - DNA, 22 bp, Artificial sequence
  Synthetic
tccgaaattgattctaattggc

SEQ ID NO: 25 - DNA, 21 bp, Artificial sequence
  Synthetic
agagaatggatactcctgccc

SEQ ID NO: 26 - DNA, 20 bp, Artificial sequence
  Synthetic
accccaacaagactcaccag

SEQ ID NO: 27 - DNA, 20 bp, Artificial sequence
  Synthetic
ggccattctatgagtgaggg

SEQ ID NO: 28 - DNA, 21 bp, Artificial sequence
  Synthetic
gcaagaagctcctctttcctc

SEQ ID NO: 29 - DNA, 20 bp, Artificial sequence
  Synthetic
gatggattttgtgaacgtgg

SEQ ID NO: 30 - DNA, 19 bp, Artificial sequence
  Synthetic
accaagaagtggctctggg

SEQ ID NO: 31 - DNA, 20 bp, Artificial sequence
  Synthetic
ccatgttgcacagaactgac

SEQ ID NO: 32 - DNA, 26 bp, Artificial sequence
  Synthetic
agacagaactacatacaaaacaatgg

SEQ ID NO: 33 - DNA, 20 bp, Artificial sequence
  Synthetic
ttttggggattgaattctgg

SEQ ID NO: 34 - DNA, 19 bp, Artificial sequence
  Synthetic
tcacggtctgctccaaatg

SEQ ID NO: 35 - DNA, 19 bp, Artificial sequence
  Synthetic
gtgacggttgctccatgtg

SEQ ID NO: 36 - DNA, 20 bp, Artificial sequence
  Synthetic
cctgagccactcagaccatc

SEQ ID NO: 37 - DNA, 20 bp, Artificial sequence
  Synthetic
agcatagcccctgagagcag

SEQ ID NO: 38 - DNA, 20 bp, Artificial sequence
  Synthetic
ataaatatgccccaacgcac

SEQ ID NO: 39 - DNA, 27 bp, Artificial sequence
  Synthetic
gaatacttaacacatcactaggtgcag

SEQ ID NO: 40 - DNA, 19 bp, Artificial sequence
  Synthetic
gttcccaccttcccaagtc

SEQ ID NO: 41 - DNA, 21 bp, Artificial sequence
  Synthetic
ctctggatttggtttcagaca

SEQ ID NO: 42 - DNA, 21 bp, Artificial sequence
  Synthetic
ggtatgggttttctgtggttt